(19) United States
(12) Patent Application Publication
(10) Pub. No.: US 2005/0075732 A1
(43) Pub. Date: Apr. 7, 2005

(54) Prevention of dislocation of an IOL

(76) Inventor: Henry M. Israel, Bnei Brak (IL)

Correspondence Address: Dekel Patent Ltd.;Beit HaRofim, Room 27, 18 Menuha VeNahala Street, Rehovot, IL

(21) Appl. No.: 10/679,413

(22) Filed: Oct. 7, 2003

(51) Int. Cl. A61F 2/16

(52) U.S. Cl. 623/6.43; 623/6.37

(57) ABSTRACT

An intraocular lens assembly operable to be positioned in a capsular bag, the IOL assembly including a lens, a haptic connected to the lens by connecting structure, and an anti-dislocation element extending from at least one of the lens, haptic and connecting structure, the anti-dislocation element being operable, when positioned in the capsular bag and upon application of a dislocating force, to become wedged and inhibit dislocation of the lens from the capsular bag. The anti-dislocation element may be generally coplanar with the lens or tilted with respect to a plane of the lens.

### FIELD OF THE INVENTION

The present invention relates generally to intraocular lens (IOL) assemblies and particularly to an IOL with structure to prevent dislocation of the IOL, such as from the capsular bag.

### BACKGROUND OF THE INVENTION

As is well known, cataract is a condition in which the natural crystalline lens of a human becomes cloudy and/or hardened. The natural lens may be removed from the capsular bag, such as by phacoemulsification, and an intraocular lens (IOL) may be implanted in the evacuated capsular bag. The IOL may be held in the capsular bag by haptics.

Natural accommodation in a normal human eye having a normal human crystalline lens involves automatic contraction or constriction and relaxation of the ciliary muscle of the eye (and zonules controlled by the ciliary muscle) by the brain in response to looking at objects at different distances. Ciliary muscle relaxation, which is the normal state of the muscle, shapes the human crystalline lens for distant vision. Ciliary muscle contraction shapes the human crystalline lens for near vision. The brain-induced change from distant vision to near vision is referred to as accommodation.

Accommodating IOL assemblies have been developed that comprise an IOL that moves in response to ciliary muscular contraction and relaxation, thereby to simulate the movement of the natural lens in the eye, and, inter alia, help provide patients with better focusing ability.

An opening is normally cut in the capsular bag, such as a continuous curvilinear capsulorhexis or other kinds of openings, in order to remove the natural lens. This weakens the bag. A possible problem of IOLs, and particularly of accommodating IOLs which may move axially along the anterior-posterior ocular axis, is the possibility of the IOL becoming dislodged or dislocated from the capsular bag.

### SUMMARY OF THE INVENTION

The present invention seeks to provide an improved IOL assembly with structure to prevent dislocation of the IOL, such as from the capsular bag, as is described more in detail hereinbelow. The term “dislocation” encompasses any movement of the IOL beyond the bounds of the capsular bag, such as but not limited to, translatory movement (anteriorly, posteriorly or in any other direction), dislodged, twisted, rotational or other contorted movement and the like, and encompasses movement of all the IOL or only parts thereof.

There is thus provided in accordance with an embodiment of the present invention an intraocular lens assembly operable to be positioned in a capsular bag, the IOL assembly including a lens, a haptic connected to the lens by connecting structure, and an anti-dislocation element extending from at least one of the lens, haptic and connecting structure, the anti-dislocation element being operable, when positioned in the capsular bag and upon application of a dislocating force, to become wedged and inhibit dislocation of the lens from the capsular bag. The anti-dislocation element may be generally coplanar with the lens or tilted with respect to a plane of the lens.

In accordance with an embodiment of the present invention the anti-dislocation element may be rigidly or flexibly attached to at least one of the lens, haptic and connecting structure.

Further in accordance with an embodiment of the present invention the anti-dislocation element includes at least one peripheral extension extending from a periphery of the lens.

Still further in accordance with an embodiment of the present invention the anti-dislocation element includes at least one projection member that protrudes non-coplanarly from at least one of the lens, haptic and connecting structure. Additionally or alternatively, the projection member may protrude from the at least one peripheral extension.

In accordance with an embodiment of the present invention the haptic includes a plate haptic and the connecting structure includes a flexible hinge.

Further in accordance with an embodiment of the present invention the lens includes an anterior lens and a posterior lens, the haptic includes an arcuate haptic connected between the anterior lens and posterior lens, and the connecting structure includes attachment points of the haptic to the anterior lens and posterior lens.

There is also provided in accordance with an embodiment of the present invention a method for inhibiting movement of a lens of an intraocular lens assembly, the method including providing a lens and a haptic connected to the lens by connecting structure, and providing an anti-dislocation element extending from at least one of the lens, haptic and connecting structure, the anti-dislocation element being operable when positioned in the capsular bag and upon application of a dislocating force to become wedged and inhibit dislocation of the lens from the capsular bag.

### BRIEF DESCRIPTION OF THE DRAWINGS

The present invention will be understood and appreciated more fully from the following detailed description taken in conjunction with the appended drawings in which:

FIGS. 1 and 2 are simplified pictorial illustrations, front and rear views respectively, of an intraocular lens assembly comprising an anti-dislocation element, constructed and operative in accordance with an embodiment of the present invention;

FIGS. 3A and 3B are simplified illustrations of an IOL assembly of the prior art without an anti-dislocation element, respectively before and after dislocation from a capsular bag;

FIG. 4 is a simplified pictorial illustration of the IOL assembly of FIGS. 1 and 2, showing the anti-dislocation element inhibiting dislocation of the IOL assembly from the capsular bag, in accordance with another embodiment of the present invention; and

FIGS. 5 and 6 are simplified pictorial illustrations of exemplary IOL assemblies, constructed and operative in accordance with other embodiments of the present invention.

### DETAILED DESCRIPTION OF THE PRESENT INVENTION

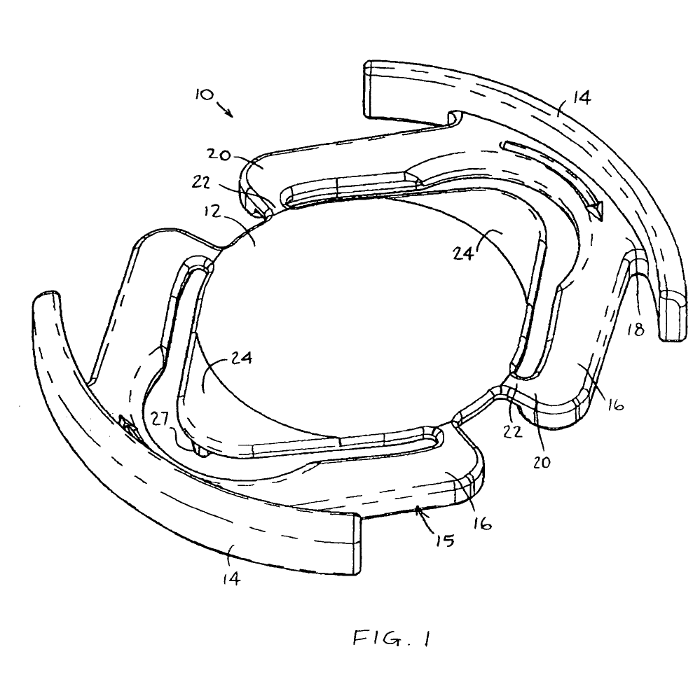

Reference is now made to FIGS. 1 and 2, which illustrate an IOL assembly 10, constructed and operative in accordance with an embodiment of the present invention.

IOL assembly 10 may be constructed with elements of the IOL assembly described in U.S. Pat. No. 6,524,340 to Israel. IOL assembly 10 may comprise a lens 12 and one or more haptics 14 connected to lens 12 by connecting structure 15. In the illustrated embodiment, IOL assembly 10 is an accommodating IOL assembly with two haptics 14 shaped generally like partial rings. It is understood that this is just one example of a suitably shaped haptic and other sizes and shapes of haptics may be used as well. In this embodiment, the connecting structure 15 comprises one or more leverage arms 16 that connect lens 12 to haptic 14. In the illustrated embodiment, there is a pair of leverage arms 16 symmetric about lens 12. Alternatively, the connecting structure 15 may simply comprise, without limitation, attachment points (flexible or rigid) of the haptics 14 to the lens 12, for example, or any other suitable structure.

IOL assembly 10 may be constructed of a clear, transparent, biologically compatible material, such as but not limited to, polymethylmethacrylate (PMMA), silicone, silicone rubber, collagen, hydrogel, hyaluronic acid (including the sodium, potassium and other salts thereof), polysulfones, thermolabile materials and other relatively hard or relatively soft and flexible biologically inert optical materials.

Each leverage arm 16 may be configured generally as a plate. A first end 18 of leverage arm 16 may be rigidly or flexibly attached to haptic 14 and a second end 20 may be rigidly or flexibly attached to the perimeter of lens 12. The second end 20 of leverage arm 16 may include two attachment zones 22, which are generally symmetric about lens 12.

Each leverage arm 16 is adapted to apply a lever force on lens 12. Specifically, each leverage arm 16 acts as a torque or moment transfer device that transfers ciliary muscle relaxation or contraction into a force on lens 12, which causes lens 12 to generally translate either anteriorly for near vision (generally as a result of ciliary muscle contraction) or posteriorly for distant vision (ciliary muscle relaxation), generally along an anterior-posterior ocular axis.

In accordance with an embodiment of the present invention, IOL assembly 10 may comprise an anti-dislocation element 24. In the illustrated embodiment, anti-dislocation element 24 comprises one or more peripheral extensions 26 extending from a periphery of lens 12. Peripheral extensions 26 may have any shape, such as but not limited to, wing-like. One or more projection members 27 (e.g., lugs, ridges, knobs and the like) may protrude from peripheral extensions 26, such as, but not necessarily, perpendicularly therefrom. Projection members 27 may also serve as anti-dislocation elements 24. Instead of protruding from peripheral extensions 26, projection members 27 may protrude non-coplanarly from lens 12, haptics 14 or from connecting structure 15, as shown in phantom lines in FIG. 2.

Anti-dislocation element 24 may be manufactured as one piece with the rest of IOL assembly 10, or may be separately made and attached to IOL assembly 10.

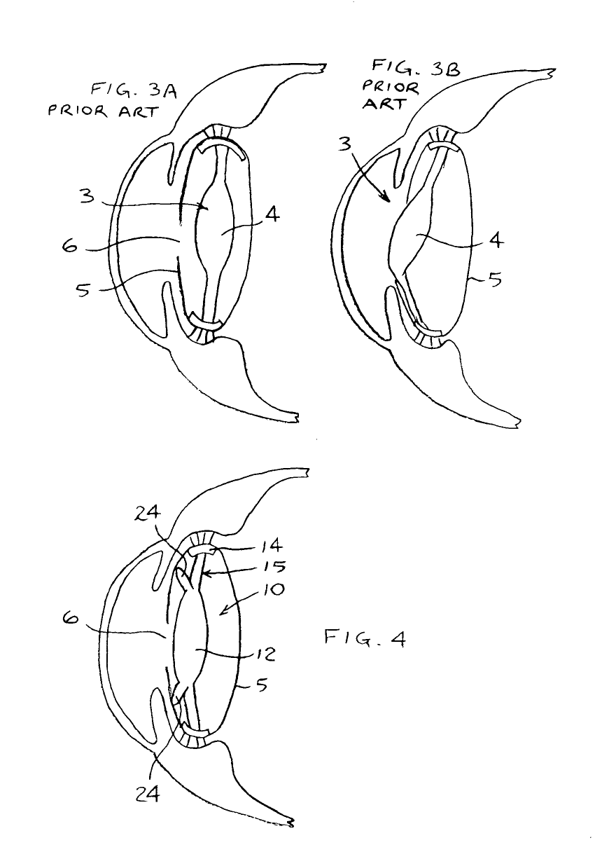

An outer edge of peripheral extensions 26 may or may not be tangent with the periphery of lens 12. Peripheral extensions 26 may or may not be symmetric about lens 12 and may extend from any portion thereof. Alternatively, peripheral extensions 26 may extend from haptics 14 or from connecting structure 15. Peripheral extensions 26 may be generally coplanar with lens 12 (FIGS. 1 and 2) or may be tilted with respect to the plane of lens 12 (as seen below in FIG. 4). Anti-dislocation element 24 may be rigidly or flexibly attached to the structure of IOL assembly 10.

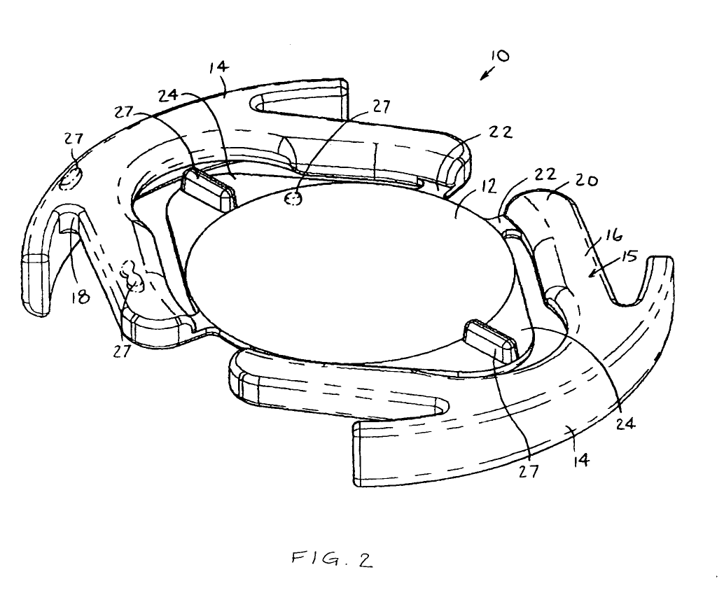

Reference is now made to FIGS. 3A and 3B, which illustrate an IOL assembly 3 of the prior art without anti-dislocation element 24. In FIG. 3A, the lens 4 of the IOL assembly 3 is shown in a capsular bag 5, capsular bag 5 having an anterior aperture 6 cut therein. In FIG. 3B, the lens 4 of the IOL assembly 3 has been dislocated in an anterior direction from the capsular bag 5, such as due to strenuous bodily activity or accommodative movement of the eye structure.

Reference is now made to FIG. 4, which illustrates IOL assembly 10 with anti-dislocation element 24 of the present invention. Upon application of a dislocating force, e.g., due to the same strenuous bodily activity or accommodative movement of the eye structure as in FIG. 3B, in the embodiment of FIG. 4, IOL assembly 10 merely translates anteriorly but is constrained to remain in the capsular bag 5, due to anti-dislocation element 24 becoming wedged. By “becoming wedged” it is meant that anti-dislocation element 24 abuts against some opposing structure or is sandwiched between opposing structures. For example, anti-dislocation element 24 may become wedged against some part of IOL assembly 10 and/or abut against some eye structure, e.g., the inner anterior wall of capsular bag 5. Anti-dislocation element 24 (e.g., projection members 27) may thus act as a wedge to prevent IOL assembly 10 from dislocating out of the capsular bag 5.

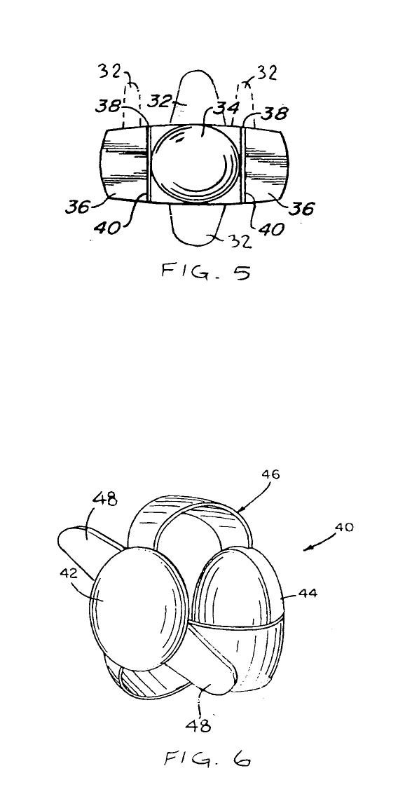

The anti-dislocation element 24 of the present invention may be implemented in any IOL assembly. For example, reference is now made to FIG. 5, which illustrates an exemplary IOL assembly 30, constructed and operative in accordance with another embodiment of the present invention. IOL assembly 30 may be constructed with elements of the IOL assembly described in U.S. Pat. No. 5,476,514 to Cumming, which may or may not provide some accommodation.

IOL assembly 30 may comprise a lens 34 and plate haptics 36 extending from diametrically opposite edges of lens 34. Haptics 36 may have inner ends joined to lens 34 and opposite outer free ends. Outer ends of haptics 36 may move anteriorly and posteriorly relative to lens 34. The particular lens embodiment illustrated may be constructed of a resilient semi-rigid material and may have flexible hinges 38 (that is, the connecting structure) which join the inner ends of the haptics 36 to the lens 34. Haptics 36 may be relatively rigid, and may be flexible about hinges 38 anteriorly and posteriorly relative to lens 34. Hinges 38 may be formed by grooves 40 on either side of IOL assembly 30. Alternatively, hinges 38 may be eliminated, and haptics 36 may be made flexible throughout their length.

In accordance with an embodiment of the present invention, an anti-dislocation element 32 may extend from lens 34. Alternatively, as shown in broken lines, anti-dislocation element 32 may extend from one or both haptics 36 or the connecting structure, i.e., hinges 38. As described similarly above for the embodiment of FIGS. 1 and 2, anti-dislocation element 32 is operable, when positioned in the capsular bag and upon application of a dislocating force, to become wedged and inhibit dislocation of lens 34 from the capsular bag.

Reference is now made to FIG. 6, which illustrates another exemplary IOL assembly 40, constructed and operative in accordance with yet another embodiment of the present invention. IOL assembly 40 may be constructed with elements of the IOL assembly described in U.S. Pat. No. 6,488,708 to Sarfarazi. IOL assembly 40 may be an open chamber intraocular lens system, which may or may not provide some accommodation. IOL assembly 40 may include an anterior lens 42, a posterior lens 44 and arcuate haptics 46 operably connecting the anterior lens 42 with the posterior lens 44 with connecting structure. (The connecting structure is the attachment points of the haptics to the lenses.) IOL assembly 40 may be elliptical in cross-section and may conform to the interior three-dimensional surface of the capsular bag.

In accordance with an embodiment of the present invention, an anti-dislocation element 48 may extend from either anterior lens 42 or posterior lens 44 or both lenses. Alternatively, as shown in phantom lines, anti-dislocation element 48 may extend from some or all haptics 46 or the connecting structure, i.e., the attachment points of the haptics to the lenses. As described similarly above for the embodiment of FIGS. 1 and 2, anti-dislocation element 48 is operable, when positioned in the capsular bag and upon application of a dislocating force, to become wedged and inhibit dislocation of lenses 42 or 44 from the capsular bag.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

1. An intraocular lens (IOL) assembly operable to be positioned in a capsular bag, the IOL assembly comprising:
a lens;
a haptic connected to said lens by connecting structure; and
an anti-dislocation element extending from at least one of said lens, haptic and connecting structure, said anti-dislocation element being operable, when positioned in the capsular bag and upon application of a dislocating force, to become wedged and inhibit dislocation of said lens from the capsular bag.

2. The intraocular lens assembly according to claim 1, wherein said anti-dislocation element is generally coplanar with said lens.

3. The intraocular lens assembly according to claim 1, wherein said anti-dislocation element is tilted with respect to a plane of said lens.

4. The intraocular lens assembly according to claim 1, wherein said anti-dislocation element is rigidly attached to at least one of said lens, haptic and connecting structure.

5. The intraocular lens assembly according to claim 1, wherein said anti-dislocation element is flexibly attached to at least one of said lens, haptic and connecting structure.

6. The intraocular lens assembly according to claim 1, wherein said anti-dislocation element comprises at least one peripheral extension extending from a periphery of said lens.

7. The intraocular lens assembly according to claim 1, wherein said anti-dislocation element comprises at least one projection member that protrudes non-coplanarly from at least one of said lens, haptic and connecting structure.

8. The intraocular lens assembly according to claim 6, further comprising at least one projection member that protrudes from said at least one peripheral extension.

9. The intraocular lens assembly according to claim 1, wherein said haptic comprises a plate haptic and said connecting structure comprises a flexible hinge.

10. The intraocular lens assembly according to claim 1, wherein said lens comprises an anterior lens and a posterior lens, said haptic comprises an arcuate haptic connected between said anterior lens and posterior lens, and said connecting structure comprises attachment points of said haptic to said anterior lens and posterior lens.

11. A method for inhibiting movement of a lens of an intraocular lens assembly, the method comprising:
providing a lens and a haptic connected to said lens by connecting structure; and
providing an anti-dislocation element extending from at least one of said lens, haptic and connecting structure, said anti-dislocation element being operable when positioned in the capsular bag and upon application of a dislocating force to become wedged and inhibit dislocation of said lens from the capsular bag.
